(19) United States
(12) Patent Application Publication
(10) Pub. No.: US 2002/0165183 A1
(43) Pub. Date: Nov. 7, 2002

(54) Methods for genetic immunization

(76) Inventors: Hans Herweijer, Madison, WI (US); Jon A. Wolff, Madison, WI (US); Aaron G. Loomis, Prairie du Sac, WI (US); Vladimir S. Trubetskoy, Madison, WI (US); Mary Kay Bates, Madison, WI (US)

Correspondence Address: Mark K. Johnson, PO Box 510644, New Berlin, WI, 53151-0644, US

(21) Appl. No.: 09/992,957

(22) Filed: Nov. 13, 2001

Related U.S. Application Data
(63) Continuation-in-part of application No. 09/450,315, filed on Nov. 29, 1999, now Pat. No. 6,379,966.
(60) Provisional application No. 60/248,275, filed on Nov. 14, 2000.

(51) Int. Cl. A61K 48/00; A61L 9/04; A61K 9/28

(52) U.S. Cl. 514/44; 424/45; 424/474

(57) ABSTRACT

The present invention relates to methods for delivering a genetic immunogen, comprising a nucleic acid capable of expressing an antigen, optionally complexed with a polymer. The nucleic acid is delivered to the host via an intravascular, oral, buccal, dermal, nasal, or rectal route, where the complex transfects lymphoid tissues resulting in expression of an encoded antigen and induction of an antigen-specific immune response.

This application is related to U.S. Provisional Application Serial No. 60/248,275 filed Nov. 14, 2000.

### FIELD OF THE INVENTION

The present invention relates to compositions and methods for transferring nucleic acids into cells in vivo for the purpose of eliciting an immune response. In preferred embodiments, the compositions include intravascular delivery systems providing high transfection efficiency; the compositions further include delivery systems providing nucleic acid transfer complexes that transfect cells with high efficiency; and methods for detection of an immune response following genetic immunization.

### BACKGROUND OF THE INVENTION

Genetic Vaccines

The development of vaccines is frequently heralded as one of the most important medical breakthroughs. Prevention of disease has provided increases in human life expectancy, lowered healthcare costs, and has resulted in enhanced quality of life. Yet more widespread use is hampered by three problems. First, it remains difficult to create effective vaccines for new microbes. Second, distribution and administration of current vaccines is expensive (requiring cooling and injection equipment). Third, parental vaccine delivery is not well accepted (discomfort with the use of needles and reactivity to adjuvant resulting in poor compliance). Genetic (or DNA) vaccines can extend the array of vaccines, and overcome these hurdles. With a classic vaccine, the antigen itself is introduced—either in the form of attenuated, killed or inactivated microbe, or as purified (recombinant) protein. With a genetic vaccine, the coding sequence for the antigen (or part of the antigen) is introduced. Following transfection of a host cell, the antigen is produced in situ.

Genetic vaccinations potentially overcome the three major hurdles listed above. By expressing antigens in vivo (e.g., after intramuscular injection of plasmid DNA expressing the antigen), one avoids the use of killed or attenuated microbes. Also, it is now possible to create vaccines for peptides that previously could not be produced or isolated. Since the full cellular biochemical machinery is available, antigens that are heavily modified can be used efficiently. A major result is the induction of strong CTL responses, where conventional subunit vaccines are skewed toward humoral responses. Since each individual vaccine requires just the coding sequence for the antigen, many different vaccines can be produced and tested for each microbe. It is even feasible to generate a shot-gun library for a given microbe, vaccinate an appropriate animal model, and determine which clones result in the greatest immunity (either humoral or cellular). Thus vastly minimizing development time. Alternatively, the expression of multiple epitopes allows genetic vaccines to better cover the variability in antigen presentation that exists in the population, due to MHC polymorphism [1]. Genetic vaccines have proven extremely efficient in eliciting immune responses against a wide variety of microbes. Protection in animal models has been demonstrated among others for influenza virus, malaria, bovine herpes virus, rabies virus, papilloma virus, herpes simplex virus, mycoplasma, and lymphocytic choriomeningitis [2, 3]. Because antigen expression is maintained over a period of time, single dose immunization may become a reality.

At the same time, genetic vaccines potentially can lower the cost per dose significantly. In appropriate form, DNA can be stored at room temperature. This would be a major advantage in developing countries. It should be noted here that the cost per genetic vaccine dose may not get below the cost per dose of vaccines that are currently in widespread use. The cost savings will come with newer vaccines for which currently no effective immunization is possible (e.g., HIV, hepatitis C, malaria), or which require frequent re-dosing (e.g., influenza).

One of the most important issues facing the vaccine field is delivery of the antigen. In the USA, the recommended schedule for children includes at least ten vaccine doses (either monovalent or combinations). That is ten or more clinic visits and needle deliveries for each child. Also, the use of influenza vaccines is increasing in an aging population, requiring yearly “flu shots.” It may be expected that immunizations to prevent milder diseases (e.g., common cold) would be very popular, provided delivery methods and associated costs become more acceptable. Delivery is the area where genetic vaccines can have the most impact, if a simple and effective oral, nasal, or dermal route can be developed. Current genetic vaccination schedules rely on two methods: (1) direct injection of naked plasmid DNA into skeletal muscle [4]; or (2) ballistic delivery of plasmid DNA into the epidermis: “gene gun.” [5] Although neither method provides any breakthrough delivery advantage over conventional vaccines, their application has proven convincingly the efficacy of the genetic vaccination concept, both in animal models and human clinical trials.

Delivery of Genetic Vaccines

It was recognized immediately that direct intramuscular injection (IM) of naked plasmid DNA could be used for vaccination purposes[4]. An early indication that expression of a transgene following naked pDNA gene transfer resulted in an immune response was observed for β-galactosidase. Expression of β-galactosidase was more prolonged in immunodeficient mice than in normal mice, following injection of pRSV-LacZ into cardiac muscle[6]. Ulmer et al., reported the first immune-protection experiments using naked pDNA delivery to skeletal muscle[7]. Following delivery of a plasmid expressing the viral nucleoprotein, a strong (CTL) immune response was measured that in subsequent experiments proved protective for influenza virus challenge (even against unrelated influenza virus strains). Since, a vast body of work has established the direct injection of pDNA into muscle as an efficient, reliable method for genetic vaccine delivery. Immune responses have been obtained for many antigens and microbes in many species, including mice, birds, fish, cattle, and monkeys (reviewed in [8]). The genetic vaccine concept has been proven in several clinical trials using intramuscular delivery. Productive immune responses were measured for HIV-1 [9] and malaria proteins [10]. Gene transfer following intramuscular injection of pDNA was found to be relatively efficient in mice, but much less so in larger rodents and primates [11]. The genetic vaccine trials have corroborated these earlier gene transfer and expression studies, by finding the need to inject large amounts of pDNA in human muscles to obtain good immune responses. This is another reason for the development of an alternative, more efficient delivery route for genetic vaccines.

Gene gun delivery aims to deposit pDNA-coated gold particles into the epidermis. The specialized APC's of the skin (Langerhans cells) reside in the epidermis-dermis boundary and are likely the real target of genetic vaccines in particle delivery systems. Tang et al. observed that following particle-mediated delivery of a human growth hormone expression plasmid an immune response was invoked [5]. This report described the first real genetic vaccination experiments, and demonstrated the potential of this technology. Subsequently, many studies have applied ballistic pDNA delivery to achieve genetic vaccination. A Phase I clinical trial is ongoing for hepatitis B virus protective immunity (PowderJect Vaccines).

Complexing pDNA with cationic liposomes (lipoplexes) has been attempted to enhance the efficiency of intramuscular delivery and for intranasal delivery. Lipoplexes may enhance the immunization efficiency by avoiding transfection of myofibers and promoting transport to draining lymph nodes [12, 13]. Innovax (Endorex/Elan) recently announced that it is developing oral (conventional) vaccines using cross-linked liposomes (Orasomes™).

Oral Delivery of Genetic Vaccines

Mathiowitz et al. described the use of biologically erodable particles to deliver small-molecule drugs and plasmid DNA [14]. Co-polymers of fumaric and sebacic acid (˜100 nm average size) could efficiently deliver insulin after gastric deposition. Low levels of β-galactosidase expression were measured in the intestines after similar pDNA particles were used. β-Galactosidase staining was mostly restricted to Peyer's patches, which also showed strong uptake of gold-labeled particles (electron microscopy studies). This is congruent with several other studies demonstrating the uptake of microspheres by Peyer's patches [15-17]. Poly(lactide-co-glycolide) (PLG) encapsulated pDNA has shown effectiveness as oral genetic vaccines, including a mucosal humoral response [18-21]. These particles appear to protect the pDNA during transit through the stomach. However, only 25% of pDNA remains active following encapsulation [18], the particles degrade slowly (and may not deliver the maximal amount at the correct intestinal location), and ligand modification is very difficult. Immune response induction requires relatively large amounts of pDNA, similar to intramuscular injection of naked pDNA [21], and more than required for gene gun delivery [22]. PLG encapsulation was developed for small drug delivery and has been shown very efficient for that purpose, as well as depositing subunit vaccines. However, these particles have not been designed from the ground up for gene transfer. Yet, these data demonstrate that orally-delivered pDNA vaccines do result in an efficient immune response, including mucosal IgA.

This was recently corroborated by Etchart et al. [23], who found a specific CTL response following introduction of measles virus haemagglutinin (HA) plasmid DNA. HA pDNA was delivered through either nasal, oral (gastric), jejunal, or buccal routes, and compared to intramuscular genetic vaccination. The best immune response with naked pDNA was observed following nasal or buccal delivery. Yet, jejunal delivery resulted in an immune response almost comparable to intramuscular delivery, if combined with the liposome DOTAP. We hypothesize that the DOTAP encapsulated and protected a small proportion of the injected HA pDNA from rapid enzymatic degradation. No data were presented on the transfected cell type(s) or what area of the small intestines result in the greatest immune response. Another report describes the use of an oral genetic vaccine for immunoprophylaxis for food allergies [24]. Chitosan pDNA particles were delivered to the intestines, resulting in production of secretory IgA against a peanut allergen, and reduced anaphylaxis upon challenge. But, chitosan may not sufficiently protect pDNA from acid hydrolysis [25]. These data provide proof-of-principle for orally delivered genetic vaccines. They also suggest the importance of using pDNA particles optimized for cellular uptake.

Immune Response Following Genetic Vaccination

Subunit vaccines generally elicit a humoral response (partly because that is what they were designed and selected for). However, for many microbial diseases such antibody responses are of little protective value and do not provide long term protection. Genetic vaccines elicit both strong humoral and strong T cell responses, thus providing better memory activity against microbes such as malaria. Immune responses following genetic vaccination have been reviewed in detail (see e.g., [2, 26]). The precise mechanism by which DNA vaccines elicit an immune response is not known, although several possibilities have been discussed. Regardless of the mechanism, however, the effectiveness of DNA vaccines to produce both humoral and cellular immunity indicates that DNA is expressed after administration, with the protein or peptide product being presented as an antigen in association with either Class I or Class II proteins. The immune response can be tailored by co-expression of cytokines [27]. For instance expression of IL-12 or interferon-6 skews the response toward Th-1, whereas co-expression of IL-4 results in a Th-2 type response [28]. Many publications have recently shown the effects of co-expression of interleukins and other cytokines, which should allow for fine tuning of the immune response following administration of genetic vaccines (e.g., [27-30]).

The mechanism of immune stimulation following genetic vaccination has been difficult to delineate, especially in the case of intramuscular administration. Dermal delivery (i.e., gene gun) likely results in direct transfection of antigen presenting cells, since the dermal layers of the skin are rich in Langerhans cells and macrophages. It was reported that only a small number of skin dendritic cells was transfected, yet this led to the activation of all dendritic cells and effective T cell activation and memory maintenance [31 ]. Myofibers can present antigen on MHC-I molecules, but appear to lack the co-stimulatory signals required for productive responses. Antigen leaked from myofibers may be taken up by APC's (e.g., in the draining lymph nodes) that can subsequently provide strong stimulation (cross-priming). Alternatively, it has been suggested that small numbers of professional APC's are directly transfected and are responsible for the induction of the complete immune response [26]. CTL responses generated in bone marrow chimeras were restricted to the donor MHC haplotype, indicating that bone marrow derived cells were responsible for priming [32]. But, transplantation studies with transfected myoblasts showed that myofiber expression alone is sufficient for induction of an MHC-I restricted CTL response against the influenza virus NP protein [3]. This indicates that transfer of antigen from myogenic cells to professional APC's can occur, thus obviating a requirement for direct transfection of BM-derived cells. Yet, given the importance of professional antigen presentation, it appears more effective to optimize gene transfer to these APC'S. The current understanding of the mechanism(s) of genetic vaccination and other considerations (such as pDNA integration, effects of long term expression) have been discussed extensively in several reviews (e.g., Donnelly et al., [2]).

Interestingly, pDNA can act as its own adjuvant. It is well established now that certain sequences in bacterial DNA stimulate the immune system [33, 34]. This appears to be based on the absence of CpG methylation in bacterial DNA, whereas in mammalian DNA most CpG sequences are methylated. By inclusion of these sequences in genetic vaccines, an enhanced immune response can be induced that is skewed to Th-l [35-37]. This provides a simpler method of directing the immune response compare to inclusion of interleukin expression vectors.

Genetic Versus Conventional Vaccines

As is clear from the discussion above, genetic vaccines have numerous advantages over conventional vaccines. They allow for efficient induction of humoral and cellular responses against almost any antigen that can be expressed by transferring its coding sequence. Novel antigens can be rapidly screened, minimizing development time and cost. This is an important feature for vaccines that require frequent adaptation because of virus antigen drift. A good example is the influenza virus, where a new vaccine is required almost every year. Another example would be the rapid development of a vaccine against a biological warfare agent (e.g., anthrax). One plasmid DNA expression vector (in which different antigen genes are cloned) can be used for many vaccines, which will increase safety and lower the cost of bringing a new vaccine into the clinical practice. Since pDNA is relatively stable, especially when dried or complexed, storage of genetic vaccines does not require cooling. This is an important aspect for world wide use of vaccines. An increased use of vaccines for certain infectious diseases is a benefit for all, while it can help prevent outbreaks and spread more effectively. Genetic vaccines also open another avenue for vaccination of livestock and wildlife. Oral delivery seems most interesting for birds and fish. These represent very large numbers of animals that are currently difficult to vaccinate. Furthermore, cost control is tremendously important, requiring prices of only a few cents per dose. Oral vaccines would significantly enhance our ability to immunize wildlife. In Europe, distribution of a live, attenuated rabies vaccine and a vaccinia recombinant vaccine has significantly diminished the incidence of rabies in wild foxes, thus demonstrating the validity of this approach [38]. Delivery of antigen to the intestines results in antigen presentation through GALT cells and induction of mucosal immunity. This is of great importance since the increased production and release of IgA antibodies provides protection against microbial infection. It remains to be determined whether our oral vaccination protocol skews toward a mucosal or a peripheral immune response, or triggers a combination.

Probably no subunit vaccine (which genetic vaccines are) can provide the broad, long lived population-wide protection that is provided by attenuated live virus vaccines (e.g., polio, pox). Yet, very few of these vaccines exist and it is unlikely that many more will be developed. Although some of the existing conventional vaccines have been in use for decades, there are still safety concerns associated with their use. Both pDNA and conventional subunit vaccines require production in biological systems (bacteria or yeast). pDNA production and purification methods are constantly improved to contain fewer contaminants [39]. Analytical techniques are improved to allow detection of low-level contaminants [40].

One concern with genetic vaccines is the possibility of DNA integration into the host genome, which could lead to oncogenic transformation of cells. This has been a topic of intense discussion in the gene therapy field at a time when the gene transfer vector of choice were retroviruses. Based on theoretical considerations, and backed up by animal experiments, this risk appeared low and acceptable for treatment of life threatening diseases [41, 42]. Since pDNA does not seem to integrate [43], the risk for oncogenic transformation is even lower. Nonetheless, the FDA may delay widespread application of genetic vaccines until more safety data are available and the risks of oncogenic transformation and germ line gene transfer are better defined.

Another concern with naked DNA administrations is the generation of anti-DNA antibodies that could cause an autoimmune disorder such as systemic lupus erythematous (SLE) [44]. High titers of anti-native DNA (nDNA) are associated with SLE [45, 46]. Repetitive intramuscular injections of large amounts of pDNA (up to 12,000 μg of pDNA) neither induced anti-nuclear antibodies (ANA) nor anti-nDNA antibodies [11]. This result is consistent with a large body of experimentation trying to create a mouse model for SLE. These studies indicate that an immune response against DNA can only be elicited in normal mice (not prone to autoimmunity) if it is denatured and complexed with a protein or adjuvant [47]. Even then, the antibodies are usually against single-stranded DNA and such antibodies are poorly correlated with autoimmune disorders. In fact, a slight increase in anti-single stranded DNA but not anti-double stranded DNA antibodies were detected following repetitive subcutaneous or intramuscular injections of pDNA [48]. Mouse anti-double-stranded DNA monoclonal antibodies have been obtained from autoimmune-prone NZB mice [49] and normal mice that have “natural” autoantibodies [50]. The inability of pDNA to elicit ANA and anti-nDNA antibodies in primates suggests that this gene transfer method is unlikely to cause an autoimmune disorder in humans.

In conclusion, the benefits of genetic vaccines are great, but there are a few safety concerns that need to be cleared up during early clinical trials. We anticipate that the ongoing trials (with intramuscular or dermal DNA delivery) will demonstrate the overall effectiveness and safety of genetic vaccines.

Nasal, Oral and Dermal Delivery Routes

Current vaccine delivery relies almost exclusively on needle delivery. Although the cost of using disposable needles and syringes is reasonable and well accepted in the developed countries, this is an additional burden for developing countries. Needle delivery has one major advantage: excellent control over vaccine delivery. However, needle sticks remain painful and impede the introduction of additional vaccines, especially in the pediatric field. An alternative to needle delivery is the “gene gun” technology, which allows for dermal delivery. Ballistic delivery requires complicated and expensive equipment, making this system less than ideal.

This leads us to believe that there is a great need for simpler, safer, cheaper, and less painful delivery methods. Using DNA-particles, one can envision delivery of genetic vaccines through nasal, oral, and dermal routes. Each would consist of a stabilized DNA-particle, allowing long term storage at room temperature. For nasal delivery, a solution would be applied to the nasal epithelium (through a swab, or a spray). Dermal delivery may proceed through the application of a topical cream, which is left in place for a short time.

Oral delivery systems have been the subject of major research efforts within the pharmaceutical industry. They can be broadly divided into immediate-, sustained-, and controlled-release systems. Immediate-release systems deliver all the contained drug upon degradation at the delivery site (e.g., dissolution of a capsule in the low pH environment of the stomach). Sustained-release and controlled-release systems do so over an extended period of time, with controlled-release systems adding predictable pharmacokinetics. To predictably obtain gene transfer in the optimal intestinal region, we will use existing (non-proprietary) immediate-release systems (e.g., coated gelatin capsules). For most small molecule drugs, delivery systems can be designed that release the drug at a specified site in the gastrointestinal tract. We will use these existing oral delivery systems to deliver our pDNA particles through the esophagus and the stomach to the preferred transfection site in the small intestines.

Mucosal Immunity

The mucosal immune system provides the first barrier against a wide variety of antigens that include food particles and microbes [51]. It has to enable an exquisitely finessed immune response that is active against pathogenic organism but tolerant to certain non-pathogenic microbes. Among its unique features is its manner of antigen presentation. The gut's epithelium contain specialized cells (M cells) that transport a wide variety of antigens and particles from the luminal to sub-epithelial space where other cells (e.g., dendritic cells) process and present the antigen. Both cellular and humoral immunity can be established by antigen presentation that probably also occurs in the mucosal associated lymphoid tissue and draining lymph nodes. Given that M cells can transport a wide variety of materials including particles, it is likely that many of the DNA particles developed in the proposed studies will be similarly transported. Commensal microorganisms are not transported probably because they do not intrinsically bind to M cells or their binding is blocked by antibodies. Therefore, transport by M cells may only require binding to their membrane surface. Another site for antigen presentation (and target for gene delivery) is the intestinal epithelial cells that express MHC class I and class II molecules and, upon stimulation with cytokines, co-stimulatory molecules such as CD80. Under abnormal conditions antigens can also enter through or between epithelial cells.

After antigen entry, the essential features of mucosal immunity are similar to those of other immunity types. Antigen presenting cells take up the antigen and present its fragments on MHC class II molecules to specific T cells. The T cells proliferate and mature into effectors that stimulate antigen-specific B cells to differentiate into plasma cells. The B cells leave the mucosa and via efferent lymph flow enter draining lymph nodes where they begin to secrete antibodies. The antibody-producing B cells (blasts), plasma cells and T cells can enter the systemic blood circulation (via efferent lymphatics and the thoracic duct). Since there are no afferent lymphatics to mucosal lymphoid tissue, dissemination of the immune response to the entire mucosal tissue occurs via the bloodstream. Homing to the mucosal lymphoid tissue is meditated by binding to β7 integrins on the surface of the endothelial cells in the mucosal lymphoid tissue. Although there is some further sub-compartmentalization in the targeting, antigen presentation to one mucosa can lead to wide-spread mucosal and systemic immunization.

Another feature of the mucosal humoral immunity is its predilection for IgA production. The IgA is transcytosed by epithelial cells from the sub-mucosal space (laminal, lamina propria) into the luminal space. Mucosal immunity leads also to IgM, IgG, and IgE production. Cytokines such as TGF-β, IL-4 and IL-10 are produced by the mucosal T and epithelial intestinal cells and commit the B cells to J chain expression and IgA production. For the purposes of mucosal vaccination, cytokines and their respective genes could be co-administered with the DNA particles to steer the immune response into producing the different humoral responses. The mucosal lymphoid tissue also contains all components of the cellular immune system including lymphocytes, natural killer (NK), macrophages, mast cells and eosinophils. The intraepithelial lymphocytes (IEL) appear to be specialized cells in the mucosa. Cytotoxic responses against viral infections occur in the lamina propria.

In conclusion, this brief review of mucosal immunity indicates that mucosal vaccination should be effective against a wide variety of infectious microorganisms. One concern is that mucosal presentation of antigen can lead to immunotolerance [24]. However, specific cytokines could prevent immunotolerance or enhance tolerance for treating allergic and autoimmune disorders.

Genetic Immunization for the Generation of Antibodies

A genetically induced immune response can also be used for the purpose of analyzing or utilizing the immune response per se (and not its protective capabilities as in genetic vaccination). For example, (monoclonal) antibodies can be derived from a genetically immunized mouse and used for analytical or therapeutic purposes. Genetic immunizations have enormous advantages over conventional (protein) immunizations. By introducing a plasmid DNA expression vector into the host, the antigen is synthesized in situ. This methodology is superior to using peptide antigens, since:

Not all peptides can be synthesized

No conjugation is required

Natural epitopes are presented

Proteins are correctly modified (e.g., glycosylated)

Faster procedure

More efficient because both MHC I and II antigen presentation

Easy to screen multiple epitopes, because DNA is cheaper than peptides

Delivery of nucleic acid expression vectors to suitable immune cells at one or more time points will allow for efficient generation of an antibody response. Antibodies can subsequently be obtained directly from the immunized host (e.g., production of polyclonal antibodies by bleeding). Alternatively, monoclonal antibodies producing hybridoma cells can be made by fusing antibody producing B (plasma) cells from the immunized host (e.g., spleen cells) with myeloma cells. Antibodies can be obtained from these hybridoma cells following culture in vitro or in vivo (ascites). Alternatively, T cell clones can be generated. Genetic immunization is extremely attractive for those investigators who have difficulty purifying a given protein or synthesizing a peptide. Also, those who already have cDNAs in mammalian expressions vectors can make antibodies quickly.

Screening for Antibody Induction Following Genetic Immunization

In a typical genetic immunization experiment, the animals are injected with plasmid DNA twice (prime and boost, separated by about 3 weeks). Ten to 14 days following the boost, serum is obtained for determination of antibody titers. If a corresponding (protein) antigen is available, standard methods for antibody detection can be used (e.g., ELISA). Mirus has developed several technologies to express antigens if not readily available. The expression vector used for genetic immunization can be transfected into one or more cell lines (e.g., by in vitro transfection using Mirus' TransIT transfection reagents). One to two days following transfection, these cell express high levels of the transgene and can therefore be used to screen for antibodies. The cells can be used in situ (e.g., immunohistochemistry, flow cytometry) or extracts can be made for use in other immunological assays (e.g., Western blotting). A wide range of cell lines and primary cells can be transfected efficiently using commercially available and our proprietary transfection reagents and methods. Proper controls can be generated by transfection of unrelated transgenes. Selection of highly specific antibodies can be achieved by expression of and selection against related genes. Another method of generating large amounts of antigen involves in vivo delivery of the expression vector. For instance, one day after TransIT In Vivo delivery, very high levels of transgene expression are found in the liver. Liver cells can be isolated, or liver extracts can be prepared, and used for screening as described above. Targeting of other organs is possible if required for specific antigens. Mirus has a large ongoing program in expression vector development, allowing high level expression of transgenes in specific cell types in vivo.

Delivery of Nucleic Acids

We have described a very efficient method for plasmid DNA gene transfer into murine liver. High levels of expression in hepatocytes could be achieved after intraportal delivery of plasmid DNA vectors with up to 10% of all liver cells transfected. Gene transfer efficiency into hepatocytes is increased by high-pressure injections and by raising the osmolarity of the injection solution. This is achieved by placing a clamp at the junction of the hepatic vein and the vena cava during the portal vein injection and the use of 15% (w/v) mannitol in the injection solution. The use of fluorescently-labeled pDNA indicates that these high-pressure conditions enable the extravasation of the pDNA, perhaps through disruption of tight junctions or an increase in sinusoid fenestrac size. High volume, high pressure tail vein injections allow for very efficient delivery of pDNA to the liver (and with lower efficiency to other organs). This simple, highly efficient procedure allows for the rapid and efficient testing of novel elements in vivo, avoiding the laborious and costly production of transgenic animals. It should be noted that intravascular delivery of pDNA to the liver of larger animals (e.g., rat, dog) is also possible.

An intravascular route of administration enables a polymer or polynucleotide to be delivered to cells more evenly distributed and more efficiently expressed than direct injections. Intravascular herein means within a tubular structure called a vessel that is connected to a tissue or organ within the body. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein. Patent number (U.S. patent application Ser. No. 08/975,573) incorporated herein by reference. An administration route involving the mucosal membranes is meant to include nasal, bronchial, inhalation into the lungs, or via the eyes.

In Vivo Transfection Reagents

Previously-developed non-viral particles aggregate in physiologic solutions. The large size of these aggregates interferes with their ability to transfect cells in vivo. In addition, previously-developed non-viral particles required a net positive charge in order for the packaged DNA to be fully protected. However, particles with a net positive charge interact non-specifically with many blood and tissue components, thereby preventing their contact with target cells in vivo. It is currently not clear what particle charge (positive, neutral, and negative) is optimal for intestinal delivery. We will therefore prepare particles with different charges and test which is most advantageous for intestinal gene transfer. Finally, currently-available preparations contain a harmful excess of free polymer, which can be removed from our particles. In summary, the inability to encase DNA into virus-like, artificial particles that are neutral or negatively-charged, and that do not aggregate have greatly hampered the efficiency and thus the utility of non-viral gene therapy. This problem in constructing DNA particles has been solved by Mirus.

We have developed a new method for constructing DNA supramolecular complexes. It entails the formation of polymers on DNA, a process termed template polymerization [52]. It greatly expands the range of tools that can be used for the construction of gene transfer particles. Conceptually, it is a “nanotechnology” and a “synthetic self-assembling system.” The process mimics biologic processes of supramolecular assembly, which often involves template polymerization. The gene complexes formed by DNA template polymerization are ideal for direct, non-viral gene therapy because they do not aggregate in physiologic solution, and are small (<70 nm).

Mirus' proprietary technology allows condensation of pDNA into small particles that are stable under physiological conditions [53], see Figure below. It allows for “recharging,” i.e., the formation of negatively charged particles that do not bind non-specifically to cells in vivo. Upon cell internalization, these particles release the pDNA, allowing nuclear uptake and expression. Ligands, endosomal release-enhancing groups, nuclear localizing signals, and other moieties can be attached to these particles through simple chemistry. Altogether, this platform technology allows for highly-efficient, cell type-specific transfections in vivo. In our ongoing gene transfer research program, we have focussed on intravascular delivery. We anticipate that intra-luminal delivery of optimized particles to the intestines will result in similarly efficient transfection of intestinal cells, and most importantly for this research proposal, the transfection of GALT antigen presenting cells.

In template polymerization (TP), cationic monomers, having an inherent electrostatic attraction for DNA, are polymerized (cross-linked) along a DNA template. Chain and step polymerization processes can be used to form DNA complexes using distinct types of cationic monomers for each process. Chain polymerization involves the successive addition of monomer units to a limited number of growing polymer chains. The polymerization rate remains constant until the monomer concentration is depleted. Monomers containing vinyl, acrylate, methacrylate, acrylamide, and methacrylamide groups undergo chain polymerization. Polymerization is initiated by radical, anionic, or cationic processes. Some of these monomers are pH-sensitive and bear a positive charge only within a certain pH range.

One of Mirus' other proprietary technologies, “DNA caging,” is a specific type of TP that prevents aggregation of DNA particles by starting with macromonomers (i.e., polycations of molecular weight>10,000) [53]. This technology comprises the treatment of preformed DNA/polycation complexes with a cleavable bifunctional reagent so DNA becomes entrapped (caged) inside a cross-linked net of counter-ions. If cross-linkers bearing positive charge were used (such as bis-imido esters) the resulting complexes stay soluble even at high salt concentrations in conditions where non-caged complexes flocculate. Caged particles are stable in physiological salt but also contain labile groups that enable the particles to disassemble in cells.

Another component of Mirus' proprietary technology comprises the preparation of negatively charged (“recharged”) particles of condensed DNA by coating them with polyanions [54]. In addition, the polyanions can be designed to carry cell-specific moieties (see below) to enhance tissue targeting. Because the pDNA is caged within a polycation layer, the outside layer of polyanions cannot displace the pDNA. This procedure represents a unique opportunity to design small and negatively charged particles of condensed pDNA. In addition, excess polymer can be removed from the caged and recharged particles using size exclusion chromatography. Preliminary results indicate that these recharged particles can transfect hepatocytes in vivo as efficiently as naked DNA.

A major part of Mirus' research is focussed on the synthesis of novel polyions and polyions with ligands, forming stable pDNA particles, and evaluating these particles in vitro and in vivo for stability, targeting specificity, and transfection efficiency. Our current efforts have mainly dealt with intravascular delivery to target liver and muscle cells. We have successfully attached several ligands to polycations and polyanions (e.g., galactose, folate, transferrin). For intestinal delivery, the folate-tagged particles are especially interesting, given the high folate receptor density. Other ligands of interest are asparagus pea lectin and bacterial adhesin lectin that have been shown to preferentially bind to M cells in Peyer's patches[55], and vitamin B12.

In summary, Mirus has developed innovative methods for forming non-viral gene transfer particles.

### REFERENCES

1. A. Suhrbier. Immunol Cell Biol 75:402-408, 1997.

2. J. J. Donnelly, J. B. Ulmer, J. W. Shiver and M. A. Liu. Annual Review of Immunology 15:617-648, 1997.

3. J. B. Ulmer, R. R. Deck, C. M. Dewitt, J. I. Donnelly and M. A. Liu. Immunology 89:59-67, 1996.

4. J. Wolff, R. Malone, P. Williams, W. Chong, G. Acsadi, A. Jani and P. Felgner. Science 247:1465-1468, 1990.

5. D. C. Tang, M. DeVit and S. A. Johnston. Nature 356:152-154, 1992.

6. G. Acsadi, S. S. Jiao, A. Jani, D. Duke, P. Williams, W. Chong and J. A. Wolff. New Biol.3:71-81, 1991.

7. J. B. Ulmer, J. J. Donnelly, S. E. Parker, G. H. Rhodes, P. L. Felgner, V. J. Dwarki, S. H. Gromkowski, R. R. Deck, C. M. DeWitt. Science 259:1745-1749, 1993.

8. J. J. Donnelly, J. B. Ulmer and M. A. Liu. Life Sci 60:163-172, 1997.

9. R. Wang, D. L. Doolan, T. P. Le, R. C. Hedstrom, K. M. Coonan, Y. Charoenvit, T. R. Jones, P. Hobart, M. Margalith. Science 282:476-480, 1998.

10. R. R. MacGregor, J. D. Boyer, K. E. Ugen, K. E. Lacy, S. J. Gluckman, M. L. Bagarazzi, M. A. Chattergoon, Y. Baine, T. J. Higgins. J Infect Dis 178:92-100, 1998.

11. S. Jiao, P. Williams, R. K. Berg, B. A. Hodgeman, L. Liu, G. Repetto and J. A. Wolff. Hum. Gene Ther. 3:21-33, 1992.

12. G. Gregoriadis, R. Saffie and J. B. de Souza. FEBS Lett 402:107-110, 1997.

13. N. Ishii, J. Fukushima, T. Kaneko, E. Okada, K. Tani, S. I. Tanaka, K. Hamajima, K. Q. Xin, S. Kawamoto. AIDS Res Hum Retroviruses 13:1421-1428, 1997.

14. E. Mathiowitz, J. S. Jacob, Y. S. Jong, G. P. Carino, D. E. Chickering, P. Chaturvedi, C. A. Santos, K. Vijayaraghavan, S. Montgomery. Nature 386:410-414, 1997.

15. D. E. Bockman and M. D. Cooper. Am J Anat 136:455-477, 1973.

16. A. T. Florence, A. M. Hillery, N. Hussain and P. U. Jani. J Drug Target 3:65-70, 1995.

17. T. H. Ermak, E. P. Dougherty, H. R. Bhagat, Z. Kabok and J. Pappo. Cell Tissue Res 279:433-436, 1995.

18. D. H. Jones, S. Corris, S. McDonald, J. C. Clegg and G. H. Farrar. Vaccine 15:814-817, 1997.

19. S. C. Chen, D. H. Jones, E. F. Fynan, G. H. Farrar, J. C. Clegg, H. B. Greenberg and J. E. Herrmann. J Virol 72:5757-5761, 1998.

20. H. O. Alpar, Y. Ozsoy, J. Bowen, J. E. Eyles, B. R. Conway and E. D. Williamson. Biochem Soc Trans 25:337S, 1997.

21. H. Kaneko, I. Bednarek, A. Wierzbicki, I. Kiszka, M. Dmochowski, T. J. Wasik, Y. Kaneko and D. Kozbor. Virology 267:8-16, 2000.

22. J. E. Herrmann, S. C. Chen, D. H. Jones, A. Tinsley-Bown, E. F. Fynan, H. B. Greenberg and G. H. Farrar. Virology 259:148-153, 1999.

23. N. Etchart, R. Buckland, M. A. Liu, T. F. Wild and D. Kaiserlian. J Gen Virol 78:1577-1580, 1997.

24. K. Roy, H.-Q. Mao, S.-K. Huang and K. W. Leong. Nature Medicine 5:387-391, 1999.

25. D. Quong and R. J. Neufeld. Biotechnol Bioeng 60:124-134, 1998.

26. D. M. Pardoll and A. M. Beckerleg. Immunity 3:165-169, 1995.

27. Z. Xiang and H. C. Ertl. Immunity 2:129-135, 1995.

28. Y. H. Chow, B. L. Chiang, Y. L. Lee, W. K. Chi, W. C. Lin, Y. T. Chen and M. H. Tao. J Immunol 160:1320-1329, 1998.

29. A. Iwasaki, B. J. Stiernholm, A. K. Chan, N. L. Berinstein and B. H. Barber. J Immunol 158:4591-4601, 1997.

30. J. J. Kim, M. L. Bagarazzi, N. Trivedi, Y. Hu, K. Kazahaya, D. M. Wilson, R. Ciccarelli, M. A. Chattergoon, K. Dang. Nat Biotechnol 15:641-646, 1997.

31. O. Akbari, N. Panjwani, S. Garcia, R. Tascon, D. Lowrie and B. Stockinger. J Exp Med 189:169-178, 1999.

32. T. M. Fu, J. B. Ulmer, M. J. Caulfield, R. R. Deck, A. Friedman, S. Wang, X. Liu, J. J. Donnelly and M. A. Liu. Mol Med 3:362-371, 1997.

33. G. B. Lipford, K. Heeg and H. Wagner. Trends Microbiol 6:496-500, 1998.

34. A. M. Krieg, A. K. Yi, J. Schorr and H. L. Davis. Trends in Microbiology 6:23-27, 1998.

35. C. Leclerc, E. Deriaud, M. Rojas and R. G. Whalen. Cell Immunol 179:97-106, 1997.

36. M. Roman, E. Martin-Orozco, J. S. Goodman, M. D. Nguyen, Y. Sato, A. Ronaghy, R. S. Kombluth, D. D. Richman, D. A. Carson. Nat Med 3:849-854, 1997.

37. Y. Sato, M. Roman, H. Tighe, D. Lee, M. Corr, M. D. Nguyen, G. J. Silverman, M. Lotz, D. A. Carson. Science 273:352-354, 1996.

38. D. B. Boyle. Reprod Fertil Dev 6:393-400, 1994.

39. R. Lahijani, G. Hulley, G. Soriano, N. A. Horn and M. Marquet. Hum Gene Ther 7:1971-1980, 1996.

40. R. Lahijani, M. Duhon, E. Lusby, H. Betita and M. Marquet. Hum Gene Ther 9:1173-1180, 1998.

41. K. Cometta, R. A. Morgan and W. F. Anderson. Hum Gene Ther 2:5-14, 1991.

42. W. F. Anderson. Hum Gene Ther 4:1-2, 1993.

43. J. A. Wolff, J. J. Ludtke, G. Acsadi, P. Williams and A. Jani. Hum. Mol. Genet. 1:363-369, 1992.

44. R. M. Nakamura, C. A. Greenwald, C. L. Peebles and E. M. Tan. Amer. Soc. Clin. Pathol. 1978.

45. F. Tron and J. F. Bach. Clin. Exper. Immunol. 28:426-432, 1977.

46. T. L. Whiteside and J. A. Dixon. Amer. J. Clin. Pathol. 72:829-835, 1979.

47. G. S. Gilkeson, A. J. Pritchard and D. S. Pisetsky. Clin. Immunol. Immunopathol. 59:288-300, 1991.

48. A. Katsumi, N. Emi, A. Abe, Y. Hasegawa, M. Ito and H. Saito. Hum. Gene Ther. 5:1335-1339, 1994.

49. D. Eilat, M. Hochberg, J. Pumphrey and S. Rudikoff. J. Immunol. 133:489-494, 1984.

50. R. Shefner, G. Kleiner, A. Turken, L. Papazian and B. Diamond. J. Exper. Med. 173:287-296, 1991.

51. P. L. Ogra. Academic Press. San Diego, 1994

52. V. S. Trubetskoy, V. G. Budker, L. J. Hanson, P. M. Slattum, J. A. Wolff and J. E. Hagstrom. Nucleic Acids Res 26:4178-4185, 1998.

53. V. S. Trubetskoy, A. Loomis, P. M. Slattum, J. E. Hagstrom, V. G. Budker and J. A. Wolff. Bioconjug Chem 10:624-628, 1999.

54. V. S. Trubetskoy, A. Loomis, J. E. Hagstrom, V. G. Budker and J. A. Wolff. Nucleic Acids Res 27:3090-3095, 1999.

55. J. M. Irache, C. Durrer, D. Duchene and G. Ponchel. Pharm Res 13:1716-1719, 1996.

56. Hepatitis Weekly May 25:1998.

57. AIDS Weekly Plus May 18:1998.

58. D. C. Jackson, C. J. Fitzmaurice, L. E. Brown and W. Zeng. Vaccine 18:355-361, 2000.

59. M. Suter, A. M. Lew, P. Grob, G. J. Adema, M. Ackermann, K. Shortman and C. Fraefel. Proc Natl Acad Sci USA 96:12697-12702, 1999.

60. V. S. Trubetskoy, P. M. Slattum, J. E. Hagstrom, J. A. Wolff and V. G. Budker. Anal Biochem 267:309-313, 1999.

61. A. Fasano. J Pharm Sci 87:1351-1356, 1998.

### SUMMARY

The present invention provides methods for delivering antigens to the interior of a cell of a vertebrate in vivo, comprising the step of introducing a preparation comprising a pharmaceutically acceptable injectable carrier and a nucleic acid coding for the antigen into the host, whereby the nucleic acid is taken up into the interior of the cell, expresses the antigen, and the antigen is presented to the immune system of the vertebrate. Also provided is a method for introducing nucleic acids into the interior of a cell of a vertebrate in vivo, whereby the nucleic acid is delivered intravascular.

A variety of cell types may be transfected with high efficiency using the compositions and methods of the present invention, including, but not limited to, cells in the liver, spleen, heart, lymph nodes, muscle, lung, thymus, kidney, skin, pancreas, intestines, mucosal cells, antigen presenting cells, T cells, B cells, macrophages. As demonstrated by the data herein, the genetic immunization methods of the present invention provides substantially higher immune response efficiencies than available systems.

The nucleic acid may code for an immunogenic peptide that is expressed by the transfected cells and which generates an immune response, thereby immunizing the vertebrate. This provides a method for obtaining long-term immunoprotection (vaccination). It also provides a method for generating a desired immune response, yielding immune cells of interest. As demonstrated by the data herein, a desired antibody response can be induced, thus providing a method for the production of antibodies directed against nucleic acid-encoded antigens.

In another aspect of the present invention, there is provided a method for immunizing a vertebrate, comprising the steps of obtaining a preparation comprising an expressible nucleic acid encoding an antigen, and introducing the preparation into a vertebrate wherein the translation antigen product of the nucleic acid is formed by a cell of the vertebrate, which elicits an immune response against the antigen.

The cells may secrete the antigen, or it may be presented by a cell of the vertebrate in the context of the major histocompatibility antigens, thereby eliciting an immune response against the antigen. In a preferred embodiment, the method is practiced by introducing the antigen-encoding nucleic acid intravascularly. In an additional preferred embodiment, the antigen-encoding nucleic acid is introduced into the tail vein of a rodent. In an additional preferred embodiment, the antigen-encoding nucleic acid is rapidly introduced into the tail vein of a rodent in a relatively large volume of a pharmaceutically acceptable carrier, resulting a transiently elevated intravascular pressure. The nucleic acid may be introduced into tissues of the body using the injectable carrier alone.

The carrier preferably is isotonic, hypotonic, or weakly hypertonic, such as provided by a sucrose, saline, or Ringer's solution. The nucleic acid may also be introduced into the host complexed with other compounds. In one aspect of this invention, the nucleic acid is complexed with a compound by mixing the nucleic acid and a polymer to form a complex wherein the zeta potential of the complex is not positive. In another aspect of this invention, the nucleic acid is complexed with a compound by mixing the nucleic acid and a polymer to form a complex wherein the zeta potential of the complex is not positive, inserting the complex into a mammalian vessel in vivo, increasing the permeability of the vessel, passing the complex through the vessel, delivering the complex into the mammalian extravascular parenchymal cell; and expressing the nucleic acid.

The method may be used to selectively elicit a humoral immune response, a cellular immune response, or a mixture of these. In embodiments wherein the cell expresses major histocompatibility complex of Class I, and the immunogenic peptide is presented in the context of the Class I complex, the immune response is predominantly cellular and comprises the production of cytotoxic T-cells.

In one such embodiment, the immunogenic peptide is associated with a virus, is presented in the context of Class I antigens, and stimulates cytotoxic T-cells which are capable of destroying cells infected with the virus. A cytotoxic T-cell response may also be produced according the method where the nucleic acid codes for a truncated viral antigen lacking humoral epitopes.

In another of these embodiments, the immunogenic peptide is associated with a tumor, is presented in the context of Class I antigens, and stimulates cytotoxic T cells which are capable of destroying tumor cells.

In one aspect, the present invention provides a composition consisting of a nucleic acid expressing an antigen under control of regulatory sequences appropriate for the target cell and host. In another aspect, the nucleic acid is complexed with a polymer. In another aspect, the present invention provides a process of delivering a biologically active substance to a cell comprising exposing the cell to the biologically active substance in the presence of a delivery system of the present invention. In a preferred embodiment, the biologically active substance is a nucleic acid. In a preferred embodiment, the delivery system comprises injecting the nucleic acid or nucleic acid—polymer complexes intravascularly. In a preferred embodiment, the delivery system comprises injecting the nucleic acid or nucleic acid—polymer complexes intravascularly under elevated pressure. In a preferred embodiment, the delivery system comprises nucleic acid or nucleic acid—polymer complexes delivered to the intestines. In a preferred embodiment, the delivery system comprises nucleic acid or nucleic acid—polymer complexes delivered to the intestines through oral intake. In a preferred embodiment, the delivery system comprises nucleic acid or nucleic acid—polymer complexes that are protected by a coating delivered to the intestines through oral intake.

In a preferred embodiment the nucleic acid expresses an antigen. In a preferred embodiment the nucleic acid expresses an antigen encoded by a cellular gene. In a preferred embodiment the nucleic acid expresses an antigen encoded by a cellular gene of unknown function. In a preferred embodiment the nucleic acid expressed an antigen from a viral pathogen. In a preferred embodiment the nucleic acid expressed an antigen from human immunodeficiency virus, human hepatitis A virus, human hepatitis B virus, human hepatitis C virus, influenza virus, smallpox (variola) virus, or human herpes virus (type I through VIII). In a preferred embodiment the nucleic acid expressed an antigen from a bacterial pathogen. In a preferred embodiment the nucleic acid expressed an antigen from E. coli or anthrax. In a preferred embodiment, the present invention provides a process for delivering nucleic acids into lymphoid cells.

In a preferred embodiment the transferred nucleic acid expresses an antigen . In a preferred embodiment the expression of the antigen induces an immune response. In a preferred embodiment the expression of the antigen induces an antigen-specific immune response. In a preferred embodiment the antigen-specific immune response results in the formation of antigen-specific antibodies. In a preferred embodiment the antigen-specific immune response results in the formation of antigen-specific antibodies which may be obtained and purified from the blood of the host. In a preferred embodiment B cells may be obtained from the host which produce antigen-specific antibodies. In a preferred embodiment B cells may be obtained from the host which produce antigen-specific antibodies and fused with myeloma cells to create monoclonal antibody producing cells. In a preferred embodiment the genetic immunization results in the induction of an antigen-specific cellular immune response. In a preferred embodiment the genetic immunization results in the induction of antigen-specific T cells. In a preferred embodiment the genetic immunization results in the induction of antigen-specific NK cells.

In a preferred embodiment the nucleic acid is complexed with TransIT In Vivo. In a preferred embodiment the nucleic acid is delivered using a TransIT In Vivo kit. In a preferred embodiment the nucleic acid is complexed with histone proteins. In a preferred embodiment the nucleic acid is complexed with histone H1 proteins. In a preferred embodiment the nucleic acid is complexed with histone proteins and crosslinked (caged). In a preferred embodiment the nucleic acid is complexed with histone H1 and crosslinked (caged). In a preferred embodiment the nucleic acid is complexed with histone H1 and crosslinked with MC449.

In a preferred embodiment, nucleic acid encoded antigen is obtained by transfecting cells with the nucleic acid. In preferred embodiments, the antigen can subsequently be used for determining the presence, amount, and affinity of antibodies directed against it. In preferred embodiments, the antigen can remain inside the cell (e.g., immunohistochemistry, flow cytometry). In preferred embodiments, the antigen can be extracted from the cell (e.g., lysis of the cell). In preferred embodiments, the cell lysate can be used in Western blotting assays. In preferred embodiments, the cell lysate can be used in immunological assays known to those skilled in the art. In preferred embodiments, the antigen can be purified from the cell or cell extract (e.g., via tags encoded in the nucleic acid to form fusion proteins with the antigen). In a preferred embodiment nucleic acid encoded antigen is obtained by transient transfection of cells in vitro. In a preferred embodiment nucleic acid encoded antigen is obtained by transient transfection of cells in vitro using a transfection reagent. In a preferred embodiment nucleic acid encoded antigen is obtained by transient transfection of cells in vivo. In a preferred embodiment nucleic acid encoded antigen is obtained by transient transfection of cells in vivo using intravascular delivery of nucleic acids. In a preferred embodiment nucleic acid encoded antigen is obtained by transient transfection of cells in vitro, and using transgene expressing cells in immunohistochemistry assays. In a preferred embodiment nucleic acid encoded antigen is obtained by transient transfection of cells in vitro, and using transgene expressing cells in flow cytometry assays. In a preferred embodiment nucleic acid encoded antigen is obtained by transient transfection of cells in vitro, and isolating proteins from the transfected cells at a predetermined time after the transfection. In a preferred embodiment nucleic acid encoded antigen is obtained by in vitro translation.

In a preferred embodiment we describe a genetic immunization method for inducing an antigen-specific immune response. The method consists of a nucleic acid sequence encoding a peptide containing at least one antigenic determinant, operatively linked to one or more control sequences such that the nucleic acid sequence is expressed in a host cell. The nucleic acid sequence is optionally formulated into a particle by complexation with one or more polymers. The nucleic acid is delivered to a vertebrate host cell.

In another preferred embodiment we describe a genetic immunization composition formulated for inducing an antigen-specific immune response. A nucleic acid sequence encoding a peptide contains at least one antigenic determinant, operatively linked to one or more control sequences such that the nucleic acid sequence is expressible in a host cell. The nucleic acid sequence is optionally formulated into a particle by complexation with a polymer, for delivery to a vertebrate host cell.

In another preferred embodiment we describe a method for generating an antibody response in a vertebrate host. We administer a nucleic acid encoding an antigen, the nucleic acid optionally being complexed to a polymer, in an amount sufficient to induce the desired immune response directed against the expressed antigen.

In another preferred embodiment we describe a method for generating an immune response in a vertebrate host. We administer a nucleic acid encoding an antigen, the nucleic acid optionally being complexed to a polymer, in an amount sufficient to induce the desired immune response directed against the expressed antigen. The nucleic acid is delivered to the intestinal lumen.

In another preferred embodiment we describe a kit for genetic immunization and detection of a genetic immune response. The kit consists of transfection complexes for in vivo and in vitro gene transfer.

### BRIEF DESCRIPTION OF THE DRAWINGS

FIG. 1: Staining of human dystrophin expressing myofibers in mar mice (1:40 serum dilution; FITC-labeled goat-anti-mouse IgG secondary). The mice were injected with a human dystrophin expression vector (CMV promoter) one week before sacrifice. The anti-human dystrophin antibody was generated by genetic immunization in ICR mice (intravascular delivery of plasmid DNA into the tail vein).

FIG. 2: Western blotting detection of luciferase antigen generated in cell lines with an antibody raised by genetic immunization. LacZ and Luciferase transfected 293 (left) and Hepa (right) cells were loaded in three concentrations (2, 6, and 10×10⁴ cells). Following electrophoresis and blotting, membranes were incubated with a 1;2,000 dilution of serum collected from a pCI-Luc immunized mouse (intravascular delivery of plasmid DNA).

### DETAILED DESCRIPTION OF THE INVENTION

### I. Definitions

To facilitate an understanding of the present invention, a number of terms and phrases are defined below:

The term “nucleic acid” is a term of art that refers to a polymer containing at least two nucleotides. “Nucleotides” contain a sugar deoxyribose (DNA) or ribose (RNA), a base, and a phosphate group. Nucleotides are the monomeric units of nucleic acid polymers. Nucleotides are linked together through the phosphate groups to form nucleic acid. A “polynucleotide” is distinguished here from an “oligonucleotide” by containing more than 100 monomeric units; oligonucleotides contain from 2 to 100 nucleotides. “Bases” include purines and pyrimidines, which further include natural compounds adenine, thymine, guanine, cytosine, uracil, inosine, and other natural analogs, and synthetic derivatives of purines and pyrimidines, which include, but are not limited to, modifications which place new reactive groups such as, but not limited to, amines, alcohols, thiols, carboxylates, and alkylhalides. The term nucleic acid includes deoxyribonucleic acid (“DNA”) and ribonucleic acid (“RNA”). The term nucleic acid encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

Nucleic acids may be linear, circular, or have higher orders of topology (e.g., supercoiled plasmid DNA). DNA may be in the form of anti-sense, plasmid DNA, parts of a plasmid DNA, vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, (interfering) double stranded RNA, short interfering RNA (siRNA), ribozymes, chimeric sequences, or derivatives of these groups. “Anti-sense” is a nucleic acid that interferes with the function of DNA and/or RNA. This may result in suppression of expression. Interfering RNA (“RNAi”) is double stranded RNA that results in catalytic degradation of specific mRNAs, and can also be used to lower gene expression. Short interfering RNA (“siRNA”) is double stranded RNA of length shorter than 30 nucleotides that results in catalytic degradation of specific mRNAs, and can also be used to lower gene expression. Natural nucleic acids have a phosphate backbone; artificial nucleic acids may contain other types of backbones, nucleotides, or bases. Artificial nucleic acids with modified backbones include peptide nucleic acids (PNAs), phosphothionates, phosphorothioates, phosphorodiamidate morpholino, and other variants of the phosphate backbone of native nucleic acids.

Examples of modified nucleotides include methylation, mustard addition, and aromatic nitrogen mustard addition. “Mustards” include nitrogen mustards and sulfur mustards. Mustards are molecules consisting of a nucleophile and a leaving group separated by an ethylene bridge. After internal attack of the nucleophile on the carbon bearing the leaving group a strained three membered group is formed. This strained ring (in the case of nitrogen mustards an aziridine ring is formed) is very susceptible to nucleophilic attack. Thus allowing mustards to alkylate weak nucleophiles such as nucleic acids. Mustards can have one of the ethylene bridged leaving groups attached to the nucleophile, these molecules are sometimes referred to as half-mustards; or they can have two of the ethylene bridged leaving groups attached to the nucleophile, these molecules can be referred to as bis-mustards. A “nitrogen mustard” is a molecule that contains a nitrogen atom and a leaving group separated by an ethylene bridge, i.e. R₂NCH₂CH₂X where R=any chemical group, and X=a leaving group typically a halogen. An “aromatic nitrogen mustard” is represented by RR′NCH₂CH₂X (wherein R=any chemical group, N=nitrogen, X=a leaving group, typically a halogen, R′=an aromatic ring, R=any chemical group).

Nucleic acid may be single (“ssDNA”), double (“dsDNA”), triple (“tsDNA”), or quadruple (“qsDNA”) stranded DNA, and single stranded RNA (“RNA”) or double stranded RNA (“dsRNA”). “Multistranded” nucleic acid contains two or more strands and can be either homogeneous as in double stranded DNA, or heterogeneous, as in DNA/RNA hybrids. Multistranded nucleic acid can be full length multistranded, or partially multistranded. It may further contain several regions with different numbers of nucleic acid strands. Partially single stranded DNA is considered a sub-group of ssDNA and contains one or more single stranded regions as well as one or more multiple stranded regions.

“Preparation of single stranded nucleic acid”: Single stranded nucleic acids can be generated by a variety of means, including denaturation, separation, chemical synthesis, isolation from viruses, enzymatic reaction. “Denaturation” is the process in which multi-stranded nucleic acid is completely or partially separated into single stranded nucleic acids. This can proceed through heating, alkaline treatment, or the addition of chemicals such as chaotropic salts or organic solvents (e.g., formamide). A mixture of nucleic acids can be “separated” by physical means such as density gradient centrifugation, gel electrophoresis, or affinity purification. Affinity purification can be accomplished by incorporating a ligand in the nucleic acid (e.g., biotin), and using the corresponding ligate (e.g., strepavidin) bound to a matrix (e.g., magnetic beads) to specifically bind and purify this nucleic acid. “Chemical synthesis” refers to the process where a single stranded nucleic acid is formed by repetitively attaching a nucleotide to the end of an existing nucleic acid. The existing nucleic acid can be a single nucleotide. Single stranded oligonucleotides can be chemically linked together to form long nucleic acids. “Viral” nucleic acids are isolated from viruses. These viruses can infect prokaryotes (e.g., M13, T7, lambda) or eukaryotes (e.g., adeno-associated virus [AAV], adenovirus, retrovirus, herpesvirus, Sindbis virus). Isolation from single stranded DNA viruses (Families of Hepadnaviridae, Circoviridae, Parvoviridae, Inoviridae, Microviridae, and Geminiviridae) will directly generate (partially) single stranded DNA.

“Enzymatic reaction” refers to processes mediated by enzymes. One strand of a double stranded nucleic acid can be preferentially degraded into nucleotides using a nuclease. Many ribonucleases are known with specific activity profiles that can be used for such a process. For instance, RNase H can be used to specifically degrade the RNA strand of an RNA-DNA double stranded hybrid nucleic acid, which in itself may have been formed by the enzymatic reaction of reverse transcriptase synthesizing the DNA stranded using the RNA strand as the template. Following the introduction of a nick, a ribonuclease can specifically degrade the strand with the nick, generating a partially single stranded nucleic acid. A RNA or DNA dependent DNA polymerase can synthesize new DNA which can subsequently be isolated (e.g., by denaturation followed by separation). The polymerase chain reaction process can be used to generate nucleic acids. Formation of single stranded nucleic acid can be favored by adding one oligonucleotide primer in excess over the other primer (“asymmetric PCR”). Alternatively, one of the DNA strands formed in the PCR process may be separated from the other (e.g., by using a ligand in one of the primers).

“Expression cassette” refers to a natural or recombinantly produced nucleic acid molecule that is capable of expressing protein(s). A DNA expression cassette typically includes a promoter (allowing transcription initiation), and a sequence encoding one or more proteins. Optionally, the expression cassette may include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. A nucleic acid can be used to modify the genomic or extrachromosomal DNA sequences. This can be achieved by delivering a nucleic acid that is expressed. Alternatively, the nucleic acid can effect a change in the DNA or RNA sequence of the target cell. This can be achieved by hybridization, multistrand nucleic acid formation, homologous recombination, gene conversion, or other yet to be described mechanisms.

The term “gene” generally refers to a nucleic acid sequence that comprises coding sequences necessary for the production of a therapeutic nucleic acid (e.g., ribozyme) or a polypeptide or precursor (e.g., factor IX). The polypeptide can be encoded by a full length coding sequence or by any portion of the coding sequence so long as the desired activity or functional properties (e.g., enzymatic activity, ligand binding, signal transduction, antigenic site) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as “5′ untranslated sequences.” The sequences that are located 3′ or downstream of the coding region and which are present on the mRNA are referred to as “3′ untranslated sequences.” The term gene encompasses both cDNA and genomic forms of a gene. A genomic form or clone of a gene contains the coding region interrupted with “non-coding sequences” termed “introns” or “intervening regions” or “intervening sequences.” Introns are segments of a gene which are transcribed into nuclear RNA. Introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, boundary elements (boundaries), matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. As used herein, the terms “an oligonucleotide having a nucleotide sequence encoding a gene,” “a molynucleotide having a nucleotide sequence encoding a gene,” and “a nucleic acid having a nucleotide sequence encoding a gene,” mean a nucleic acid sequence comprising the coding region of a gene or in other words the nucleic acid sequence which encodes a gene product. The coding region may be present in either a cDNA, genomic DNA or RNA form. When present in a DNA form, the nucleic acid may be single-stranded, double-stranded, multistranded, partially single stranded, or partially multistranded. Suitable control elements such as, but not limited to, enhancers/promoters, splice junctions, and polyadenylation signals, may be placed in close proximity to the coding region of the gene if needed to permit proper initiation of transcription and correct processing of the primary RNA transcript. Alternatively, the coding region utilized in the expression vectors may contain endogenous enhancers/promoters, splice junctions, intervening sequences, polyadenylation signals; exogenous control elements; or a combination of both endogenous and exogenous control elements.

The term “isolated” when used in relation to a nucleic acid, as in “an isolated nucleic acid” refers to a nucleic acid sequence that is identified and separated from at least one contaminant nucleic acid with which it is ordinarily associated in its natural source. Isolated nucleic acid is such present in a form or setting that is different from that in which it is found in nature. In contrast, “non-isolated nucleic acids” are nucleic acids, such as DNA and RNA, found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid may be present in single stranded, partially single stranded, multistranded, or partially multistranded form.

As used herein, the term “gene expression” refers to the process of converting genetic information encoded in a gene into RNA (e.g., mRNA, rRNA, tRNA, or snRNA) through “transcription” of a deoxyribonucleic gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Two molecules are combined, to form a “complex” through a process called “complexation” or “complex formation,” if the are in contact with one another through “non-covalent” interactions such as, but not limited to, electrostatic interactions, hydrogen bonding interactions, and hydrophobic interactions. An “interpolvelectrolyte complex” is a non-covalent interaction between polyelectrolytes of opposite charge. A molecule is “modified,” through a process called “modification,” by a second molecule if the two become bonded through a covalent bond. That is, the two molecules form a covalent bond between an atom form one molecule and an atom from the second molecule resulting in the formation of a new single molecule. A chemical “covalent bond” is an interaction, bond, between two atoms in which there is a sharing of electron density.

The terms “naked nucleic acid” and “naked polynucleotide” indicate that the nucleic acid or polynucleotide is not associated with a transfection reagent or other delivery vehicle that is required for the nucleic acid or polynucleotide to be delivered to the cell. A “transfection reagent” is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include cationic liposomes and lipids, polyamines, calcium phosphate precipitates, histone proteins, polyethylenimine, and polylysine complexes. It has been shown that cationic proteins like histones and protamines, or synthetic polymers like polylysine, polyarginine, polyornithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via ligands that bind to receptors in the cell. For example, cationic liposomes or polylysine complexes have net positive charges that enable them to bind to DNA or RNA. Polyethylenimine, which facilitates gene transfer without additional treatments, probably disrupts endosomal function itself.

Other vehicles are also used, in the prior art, to transfer genes into cells. These include complexing the nucleic acids on particles that are then accelerated into the cell. This is termed “biolistic” or “gn” techniques. Other methods include electroporation, microinjection, liposome fusion, protoplast fusion, viral infection, and iontophoresis.

“Intravascular” refers to an intravascular route of administration that enables a polymer, oligonucleotide, or polynucleotide to be delivered to cells more evenly distributed and more efficiently than direct injections. Intravascular herein means within an internal tubular structure called a vessel that is connected to a tissue or organ within the body of an animal, including mammals. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein. “Intracoronary” refers to an intravascular route for delivery to the heart wherein the blood vessels are the coronary arteries and veins.

Delivery of a nucleic acid means to transfer a nucleic acid from a container outside an animal to near or within the outer cell membrane of a cell in the animal. The term “transfection” is used herein, in general, as a substitute for the term “delivery,” or, more specifically, the transfer of a nucleic acid from directly outside a cell membrane to within the cell membrane. If the nucleic acid is a primary RNA transcript that is processed into messenger RNA, a ribosome translates the messenger RNA to produce a protein within the cytoplasm. If the nucleic acid is a DNA, it enters the nucleus where it is transcribed into a messenger RNA that is transported into the cytoplasm where it is translated into a protein. Therefore, if a nucleic acid expresses its cognate protein, then it must have entered a cell. A protein may subsequently be degraded into peptides, which may be presented to the immune system.

A “therapeutic gene” refers herein to a nucleic acid that may have a therapeutic effect upon transfection into a cell. This effect can be mediated by the nucleic acid itself (e.g., anti-sense nucleic acid), following transcription (e.g., anti-sense RNA, ribozymes, interfering dsRNA, siRNA), or following expression into a protein. “Protein” refers herein to a linear series of greater than 2 amino acid residues connected one to another as in a polypeptide. A “therapeutic” effect of the protein in attenuating or preventing the disease state can be accomplished by the protein either staying within the cell, remaining attached to the cell in the membrane, or being secreted and dissociated from the cell where it can enter the general circulation and blood. Secreted proteins that can be therapeutic include hormones, cytokines, growth factors, clotting factors, anti-protease proteins (e.g., alpha1-antitrypsin), angiogenic proteins (e.g., vascular endothelial growth factor, fibroblast growth factors), antiangiogenic proteins (e.g., endostatin, angiostatin), and other proteins that are present in the blood. Proteins on the membrane can have a therapeutic effect by providing a receptor for the cell to take up a protein or lipoprotein. Therapeutic proteins that stay within the cell (“intracellular proteins”) can be enzymes that clear a circulating toxic metabolite as in phenylketonuria. They can also cause a cancer cell to be less proliferative or cancerous (e.g., less metastatic), or interfere with the replication of a virus. Intracellular proteins can be part of the cytoskeleton (e.g., actin, dystrophin, myosins, sarcoglycans, dystroglycans) and thus have a therapeutic effect in cardiomyopathies and musculoskeletal diseases (e.g., Duchenne muscular dystrophy, limb-girdle disease). Other therapeutic proteins of particular interest to treating heart disease include polypeptides affecting cardiac contractility (e.g., calcium and sodium channels), inhibitors of restenosis (e.g., nitric oxide synthetase), angiogenic factors, and anti-angiogenic factors.

The term “antigen” is defined as anything that can serve as a target for an immune response. The term “adjuvant” means compounds that, when used in combination with specific antigens, augment or otherwise alter or modify the resultant immune responses. The term “vaccine” is defined herein as a suspension or solution of one or more antigenic moieties, or nucleic acids capable of directing the synthesis of one or more antigenic moieties, which is delivered into an organism to produce an immune response. The “antigenic moiety” can be either a live or killed microorganism, or a natural product purified from a microorganism or other cell including, but not limited to tumor cells, a synthetic product, a genetically engineered protein, peptide, polysaccharide or similar product or an allergen. The antigenic moiety can also be a subunit of a protein, peptide, polysaccharide or similar product. The term “cell mediated immunity” is defined as an immune response mediated by cells or the products they produce, such as cytokines, rather than by antibody. It includes, but is not limited to, delayed type hypersensitivity and cytotoxic T cells. The term “humoral immunity” relates to an immune response mediated by antibodies and the cells involved in the production of antibodies. Cell mediated and humoral immunity are often induced simultaneously and influence each other. Since the immune systems of all vertebrates operate similarly, the applications described can be implemented in all vertebrate systems, comprising mammalian and avian species, as well as fish. The term “adjuvant” as used herein is any substance whose admixture with an immunogen modifies the immune response. Modification of the immune response means augmentation, intensification, or broadening the specificity of either or both antibody and cellular immune responses. Modification of the immune response can also mean decreasing or suppressing certain antigen-specific immune responses such as the induction of tolerance. A “hapten” is defined herein as a substance that reacts selectively with appropriate antibodies or T cells but the hapten itself is usually not immunogenic. Most haptens are small molecules or small parts of large molecules, but some macromolecules can also function as haptens.

As used herein, the term “immunogenic protein” or “immunogenic peptide” or “immunogen” is meant to refer to an antigen that is a target for an immune response and against which an immune response can be elicited. The immunogenic protein shares at least an epitope with a protein against which immunization is desired. In one application, the immune response is directed at proteins associated with conditions, infections, diseases or disorders such as allergens, pathogen antigens, antigens associated with cancer cells or cells involved in autoimmune diseases (i.e., generally described as “genetic vaccination”). In another application, the antigen-directed immune response is applied to (basic) biological studies, the generation of cellular or humoral immune response products (e.g., CTL clones, B cells, plasma cells, antibodies), or derivatives thereof (e.g., monoclonal antibodies. This process is generally referred to as “genetic immunization” or “DNA immunization.” The immunogenic antigen is encoded by the coding sequence of a genetic construct (see vector below). As used herein, the term “sharing an epitope” refers to proteins which comprise at least one epitope that is identical to or substantially similar to an epitope of another protein. As used herein, the term “substantially similar epitope” is meant to refer to an epitope that has a structure which is not identical to an epitope of a protein but nonetheless invokes a cellular or humoral immune response which cross reacts to that protein. For vaccination purposes, the genetic vaccine is administered to the vaccinated individual, the genetic construct is taken up by the cells of the individual, the coding sequence is expressed and the immunogenic protein is produced. The immunogenic protein induces an immune response against the immunogenic protein in the individual. The immune response is directed against proteins associated with conditions, infections, diseases or disorders such as allergens, pathogen antigens, antigens associated with cancer cells or cells involved in autoimmune diseases. Thus the vaccinated individual may be immunized prophylactically or therapeutically to prevent or treat conditions, infections, diseases or disorders. The immunogenic protein refers to peptides and protein encoded by gene constructs of the present invention which act as target proteins for an immune response. The immunogenic protein shares at least an epitope with a protein from the allergen, pathogen or undesirable protein or cell-type such as a cancer cell or a cell involved in autoimmune disease against which immunization is required. The immune response directed against the immunogenic protein will protect the individual against and treat the individual for the specific infection or disease with which the protein from the allergen, pathogen or undesirable protein or cell-type is associated. The immunogenic protein does not need to be identical to the protein against which an immune response is desired. Rather, the immunogenic target protein must be capable of inducing an immune response that cross reacts to the protein against which the immune response is desired.

The term “antibody” encompasses whole immunoglobulin of any class, chimeric antibodies, hybrid antibodies with dual or multiple antigen specificities and fragments including hybrid fragments. Also included within the meaning of “antibody” are conjugates of such fragments, and so-called antigen binding proteins (single chain antibodies) as described, for example, in U.S. Pat. No. 4,704,692. Alternatively, the encoded antibodies can be anti-idiotypic antibodies (antibodies that bind other antibodies) as described, for example, in U.S. Pat. No. 4,699,880.

“Vectors” are nucleic acid molecules originating from a virus, a plasmid, or the cell of an organism into which another nucleic fragment of appropriate size can be integrated without loss of the vectors capacity for self-replication. Vectors introduce nucleic acids into host cells, where it can be reproduced. Examples are plasmids, cosmids, and yeast artificial chromosomes. Vectors are often recombinant molecules containing nucleic acid sequences from several sources. Vectors include viruses, for example adenovirus (an icosahedral (20-sided) virus that contains DNA; there are over 40 different adenovirus varieties, some of which cause respiratory disease), adeno-associated virus (AAV, a parvovirus that contains single stranded DNA), or retrovirus (any virus in the family Retroviridae that has RNA as its nucleic acid and uses the enzyme reverse transcriptase to copy its genome into the DNA and integrate into the host cell's chromosome).

The gene expression vectors used in the practice of the invention may be constructed to include coding regions for peptides of diagnostic (i.e., marker proteins), therapeutic or immunostimulatory interest. For example, a mixture of polynucleotides or separately co-administered group of polynucleotides may be of use in immunizing a host against more than one antigen and/or to further stimulate a host immune response (by, for example, including a gene operatively encoding for an immuno-suppressive cytokine such as TGF.beta. or a relevant histo-compatibility protein in the recombinant gene expression vector). The gene expression vectors of the invention may also encode peptides having more than one biological activity. For example, a polynucleotide operatively encoding for a peptide may be coupled to or administered with a polynucleotide operatively encoding an antibody in such a way that both peptide and antibody will be expressed. Further, the same vector may also encode an antigen, T cell epitope, cytokine or other polypeptides or immunostimulatory sequences in combination.

The process of delivering a nucleic acid to a cell has been commonly termed transfection or the process of “transfecting” and also it has been termed “transformation.” The term transfecting as used herein refers to the introduction of foreign DNA into cells. The nucleic acid could be used to produce a change in a cell that can be therapeutic. The delivery of nucleic acid for therapeutic and research purposes is commonly called “gene therapy.” The delivery of nucleic acid can lead to modification of the genetic material present in the target cell. The term “stable transfection” or “stably transfected” generally refers to the introduction and integration of foreign nucleic acid into the genome of the transfected cell. The term “stable transfectant” refers to a cell which has stably integrated foreign nucleic acid into the genomic DNA. Stable transfection can also be obtained by using episomal vectors that are replicated during the eukaryotic cell division (e.g., plasmid DNA vectors containing a papilloma virus origin of replication, artificial chromosomes). The term “transient transfection” or “transiently transfected” refers to the introduction of foreign nucleic acid into a cell where the foreign nucleic acid does not integrate into the genome of the transfected cell. The foreign nucleic acid persists in the nucleus of the transfected cell. The foreign nucleic acid is subject to the regulatory controls that govern the expression of endogenous genes in the chromosomes. The term “transient transfectant” refers to a cell which has taken up foreign nucleic acid but has not integrated this nucleic acid.

As used herein, the term “sample” is used in its broadest sense. Sample is meant to include a specimen or culture obtained from any source, including biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

The following abbreviations are used herein: HEPES, 4-(2-hydroxyethyl)-1-piperazineethanesulfonic acid; PBS, phosphate-buffered saline; EGTA, ethylenebis(oxyethylenenitrilo)-tetraacetic acid.

### II. The Invention

The present invention relates to compositions and methods for genetic immunization. The methods comprise delivery systems for nucleic acids in vivo. In preferred embodiments, the in vivo delivery of nucleic acids results in an immune response directed against a nucleic acid encoded antigen. The compositions comprise in vivo transfection reagent. Methods disclosed in this invention relate to the detection of a genetic immune response. In preferred embodiments, the antigen is generated in vitro (via transfection of cells) or in vivo, and subsequently used in immunological assays. Various immunoassay methods may be employed in conjunction with such antibodies, such as, for example, Western blotting, ELISA, RIA, and the like, all of which are known to those of skill in the art.

ELISAs may be used in conjunction with the invention. In an ELISA assay, proteins or peptides incorporating pathogen antigen sequences are immobilized onto a selected surface, preferably a surface exhibiting a protein affinity such as the wells of a polystyrene microtiter plate. After washing to remove incompletely adsorbed material, it is desirable to bind or coat the assay plate wells with a nonspecific protein that is known to be antigenically neutral with regard to the test antisera such as bovine serum albumin (BSA), casein or solutions of milk powder. This allows for blocking of nonspecific adsorption sites on the immobilizing surface and thus reduces the background caused by nonspecific binding of antisera onto the surface.

After binding of antigenic material to the well, coating with a non-reactive material to reduce background, and washing to remove unbound material, the immobilizing surface is contacted with the antisera or clinical or biological extract to be tested in a manner conducive to immune complex (antigen/antibody) formation. Such conditions preferably include diluting the antisera with diluents such as BSA, bovine gamma globulin (BGG) and phosphate buffered saline (PBS)/Tween. These added agents also tend to assist in the reduction of nonspecific background. The layered antisera is then allowed to incubate for from 2 to 4 hours, at temperatures preferably on the order of about 25.degree. to about 27.degree. C. Following incubation, the antisera-contacted surface is washed so as to remove non-immunocomplexed material. A preferred washing procedure includes washing with a solution such as PBS/Tween or borate buffer.

Following formation of specific immunocomplexes between the test sample and the bound antigen, and subsequent washing, the occurrence and even amount of immunocomplex formation may be determined by subjecting same to a second antibody having specificity for the first. To provide a detecting means, the second antibody will preferably have an associated enzyme that will generate a color development upon incubating with an appropriate chromogenic substrate. Thus, for example, one will desire to contact and incubate the antisera-bound surface with a urease or peroxidase-conjugated anti-human IgG for a period of time and under conditions which favor the development of immunocomplex formation (e.g., incubation for 2 hours at room temperature in a PBS-containing solution such as PBS-Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline-6-sulfonic acid [ABTS] and H.sub.2 O.sub.2, in the case of peroxidase as the enzyme label. Quantification is then achieved by measuring the degree of color generation, e.g., using a visible spectra spectrophotometer.

Immunoprecipitation may be used in conjunction with the invention. The antibodies of the present invention may be particularly useful for the isolation of antigens by immunoprecipitation. Immunoprecipitation involves the separation of the target antigen component from a complex mixture, and is used to discriminate or isolate minute amounts of protein. For the isolation of membrane proteins cells must be solubilized into detergent micelles. Nonionic salts are preferred, since other agents such as bile salts, precipitate at acid pH or in the presence of bivalent cations. In an alternative embodiment the antibodies of the present invention are useful for the close juxtaposition of two antigens. This is particularly useful for increasing the localized concentration of antigens, e.g. enzyme-substrate pairs.

The compositions of the present invention may find use in immunoblot or Western blot analysis. The anti-peptide antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. This is especially useful when the antigens studied are immunoglobulins (precluding the use of immunoglobulins binding bacterial cell wall components), the antigens studied cross-react with the detecting agent, or they migrate at the same relative molecular weight as a cross-reacting signal.

Immunologically-based detection methods for use in conjunction with Western blotting include enzymatically-, radiolabel-, or fluorescently-tagged secondary antibodies against the peptide moiety are considered to be of particular use in this regard.

Genetically immunized mice may be used to produce monoclonal antibodies. The means for preparing and characterizing antibodies are well known in the art (See, e.g., Antibodies: A Laboratory Manual, Cold Spring Harbor Laboratory, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified epitopic protein, polypeptide or peptide. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986, pp. 60-61), but mice are preferred, with the BALB/c mouse being most preferred as this is most routinely used and generally gives a higher percentage of stable fusions. Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5′10.sup.7 to 2′10.sup.8 lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp.65-66, 1986; Campbell, pp.75-83, 1984). For example, a mouse myeloma cell line that may be used is the 8-azaguanine-resistant mouse murine myeloma SP2/0 cell line.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacteriumtuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

### III. Methods of Use

A. A Process of Delivering a Biologically Active Substance to a Cell

The present invention provides a process of delivering a biologically active substance to a cell. In accordance with that process, a target cell (a cell to which the substance is to be delivered) is exposed to the biologically active substance in the presence of a delivery system of the present invention. Preferred such delivery systems are the same as set forth above. A target cell can be located in vitro (e.g., cell culture) or in vivo (e.g., in a living organism).

As used herein, the phrase “biologically active substance” means any substance having the ability to alter the function of a living cell, tissue or organism. A biologically active substance can be a drug or other therapeutic agent. A biologically active substance can also be a chemical that interacts with and alters the function of a cell. By way of example, a biologically active substance can be a protein or peptide fragment thereof such as a receptor agonist or antagonist. In addition, a biologically active substance can be a nucleic acid.

Where the target cell is located in vitro, the biologically active substance, and the delivery system are typically added to the culture medium in which the cell is being cultured. The active substance and delivery system can be added to the medium either simultaneously or sequentially. Alternatively, the biologically active substance and the delivery system can be formed into a complex and then added to the medium. A complex between a biologically active substance and a delivery system of the present invention can be made by contacting those materials under appropriate reaction conditions. Means for making such complexes are set forth hereinafter in the Examples.

Where the target cell is located in vivo, the biologically active substance and the delivery system are typically administered to the organism in such a way as to distribute those materials to the cell. The materials can be administered simultaneously or sequentially as set forth above. In one embodiment, the biologically active substance and the delivery system are administered as a complex. The delivery system and biologically active substance can be infused into the cardiovascular system (e.g., intravenously, intraarterially), injected directly into tissue containing the target cell (e.g., intramuscularly), or administered via other parenteral routes well known to one skilled in the art.

B. Process of Genetic Immunization

A variety of cell types may be transfected with high efficiency using the compositions and methods of the present invention, including, but not limited to, cells in the liver, spleen, heart, lymph nodes, muscle, lung, thymus, kidney, skin, pancreas, intestines, mucosal cells, antigen presenting cells, T cells, B cells, macrophages. Following transfection, the nucleic acid expresses the encoded antigen, resulting in the induction of an immune response. This immune response may be aimed at inducing protective immunity in the host, either prophylactic (in which case the process would be termed vaccination) or therapeutic. In other applications, the immune response may be aimed at obtained immune cells specific for the antigen, for example B cells producing antibodies. These immune cells or immune cell products, may be used for analytical or therapeutic purposes. As demonstrated by the data herein, the genetic immunization methods of the present invention provides substantially higher immune response efficiencies than available systems.

C. Process of Screening for Genetic Immunization

The invention discloses methods for the generation of antigen. The antigen can subsequently be used in many different assays for the detection of a specific immune response directed against the antigen following a genetic immunization. The lack of available antigen in genetic immunization protocols is thereby solved. Antigen can be produced in vitro by transfection of cells or by in vitro translation (or coupled transcription-translation), or in vivo.

### EXPERIMENTAL

The following examples are provided in order to demonstrate and further illustrate certain preferred embodiments and aspects of the present invention and are not to be construed as limiting the scope thereof.

### EXAMPLE 1

Intestinal cells can be transfected by injecting pDNA solutions into the mesenteric vasculature. In rats, a 3-cm section of the small intestines was clamped, blocking both vascular inflow and outflow. Two ml of saline with 100 μg pCI-Luc⁺ (an expression vector in which the luciferase gene is under transcriptional control of the cytomegalovirus promoter) were injected into the mesenteric vein. The clamps were released 2 minutes later. One day after pDNA delivery, the rats were sacrificed, and the injected section of the intestines was excised. An average luciferase expression level of 13 ng per section was measured. This shows that vascular delivery of naked pDNA is effective for the small intestines.

### EXAMPLE 2

To test if naked plasmid DNA can be taken up by intestinal cells following oral delivery, we have injected pDNA solutions into the intestinal lumen of mice. A volume of 250 μl containing 50 μg pCI-Luc⁺ was delivered into the lumen using a 1 ml syringe and a 30 g needle. Different areas of the intestines were targeted (duodenum, jejunum, ileum). One day after injection, the intestines were removed, cut in three cm sections and assayed for luciferase expression. In all cases, luciferase expression levels were highest close to the site of injection: 0.1-1 ng luciferase protein per section. These data demonstrate that naked pDNA can be taken up by intestinal cells following delivery into the lumen. It also suggests that pDNA is not degraded so rapidly as to prevent transfection.

### EXAMPLE 3

pDNA was mixed and condensed with polymers M16, M66, and M67, optionally crosslinked (using DTBP), and formulated for injection. The pDNA solution was injected as described above, with all injections into the duodenum. Luciferase expression was measured throughout the intestinal tract, with the highest levels usually in the duodenum. Expression levels were in the same range as for naked pDNA injections (0.1-1 ng per section). This demonstrates that caged particles can deliver pDNA efficiently to cells and that the crosslinking of the polymers can be reversed once inside a cell.

### EXAMPLE 4

To test the ability of pDNA particles to transfect antigen presenting cells, mice were injected in the intestinal lumen with a HBsAg expression vector (CMV promoter, 10 μg pDNA per mouse). Three weeks after intestinal delivery, serum antibodies against HBsAg were measured. Blood was collected from the retro-orbital sinus and spun down to obtain plasma. The presence of HBsAg-antibodies was determined in ten-fold serial serum dilutions using an ELISA test (as described in the Experimental Plan). As a control, direct intramuscular injections were performed. Several different types of pDNA particles were tested: naked pDNA, TransIT LT1 (3:1 and 10:1), TransIT In Vivo, caged (3 different cleavable types), and recharged (negative) particles. Recharged pDNA particles delivered to the gut showed the strongest evidence of HBsAg antibodies in the serum after 3 weeks, without boosting, and had again the highest readings one week after boosting. Interestingly, these particles also appeared to work well upon intramuscular delivery. These data demonstrate that DNA particles can deliver pDNA to antigen presenting cells and result in the induction of antibodies directed against the expressed transgene.

### EXAMPLE 5

ICR mice were immunized with pCI-Luc, an expression vector in which luciferase is under transcriptional control of the human CMV promoter. Several gene transfer routes were tested: intravascular delivery of plasmid DNA into the tail vein, intravascular delivery into the tail vein of plasmid DNA complexed with linear polyethylenimine (IPEI) and polyacrylamide (PAA), and direct IM injection of naked plasmid DNA (3-5 mice per group). All mice were boosted on day 21 using the same gene delivery method as used for the prime. While luciferase is considered to have a low immunogenicity, immunization using intravascular delivery resulted in a strong antibody response after the prime DNA delivery (see Table). As purified luciferase is readily available, as well as control antibodies, a direct ELISA was used for determination of antibody titers in mouse sera. It should be noted that only 3-5 mice were immunized and that analysis was performed once with ten-fold serial serum dilutions. Nonetheless, the results clearly show the superiority of TransIT In Vivo gene delivery for immunization purposes. This is not surprising given the large amount of antigen that is produced using this method. For instance, one day following intravascular pCI-Luc delivery, luciferase expression in the liver averages 5 μg of protein. PEI/PAA, resulting in luciferase expression predominantly in the lungs, provides a viable alternative for genetic immunization technique. In contrast, the classic injection of plasmid DNA directly into skeletal muscle is not nearly as effective at generating an antibody response.

| | | Anti-luciferase | Anti-luciferase antibody |
|---|---|---|---|
| | Sample | antibody (range | (average ng/ml relative |
| | (Immunization | of approximate | to control monoclonal |
| Day | Method) | titers) | antibody) |
| 0 | Pre-immune | 10-100 | 73 |
| 21 | Post prime (high | 100-10,000 | 3,050 |
| | pressure tail vein | | |
| | delivery) | | |
| 21 | Post prime (lPEI/PAA) | 100 | Not determined |
| 21 | Post prime (naked | 10-100 | 49 |
| | DNA, direct | | |
| | intramuscular) | | |
| 35 | Post boost (high | 100-100,000 | 106,075 |
| | pressure tail vein | | |
| | delivery) | | |
| 35 | Post boost (lPEI/PAA) | 100-1,000 | >200,000 |
| 35 | Post boost (naked | 100-1,000 | 638 |
| | DNA, direct | | |
| | intramuscular) | | |

### EXAMPLE 6

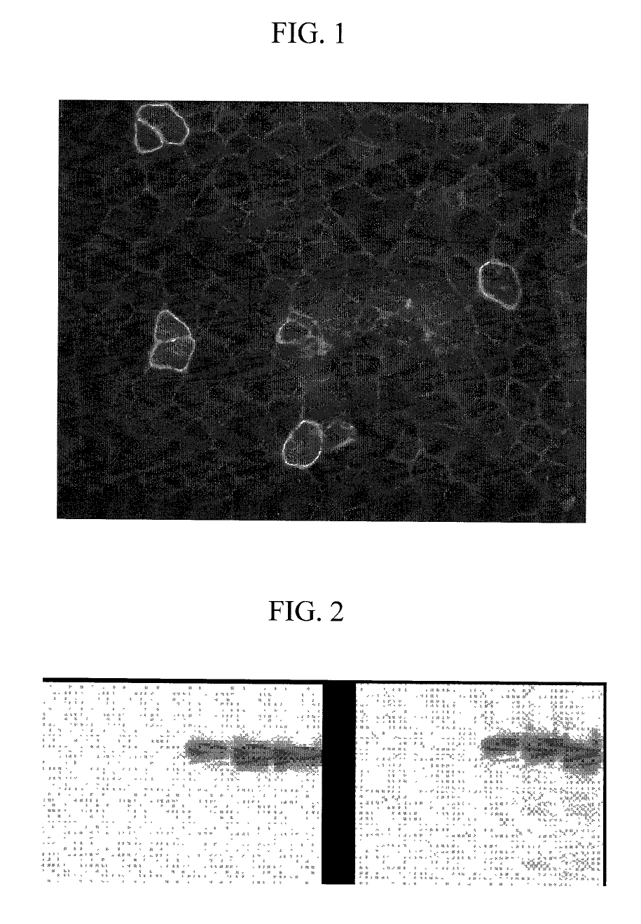

An anti-human dystrophin antibody was generated in ICR mice by genetic immunization. The mice were primed and boosted by high pressure tail vein delivery of 100 μg of a human dystrophin expression cassette (2 boosts at 2 and 3 weeks after the prime). Sera were obtained 3 days after the second boost and used to stain for human dystrophin expression in mdx (dystrophin deficient) mice previously injected with 10 μg of the same expression vector (IM). FIG. 1 shows a typical result, clearly indicating the presence of myofibers expressing human dystrophin. These results are identical to those obtained with commercially available anti-human dystrophin antibodies. This indicates that IV genetic immunization can result in the generation of antibodies against clinically relevant target proteins, and that the titers are sufficient to be used for immunohistochemistry.

### EXAMPLE 7

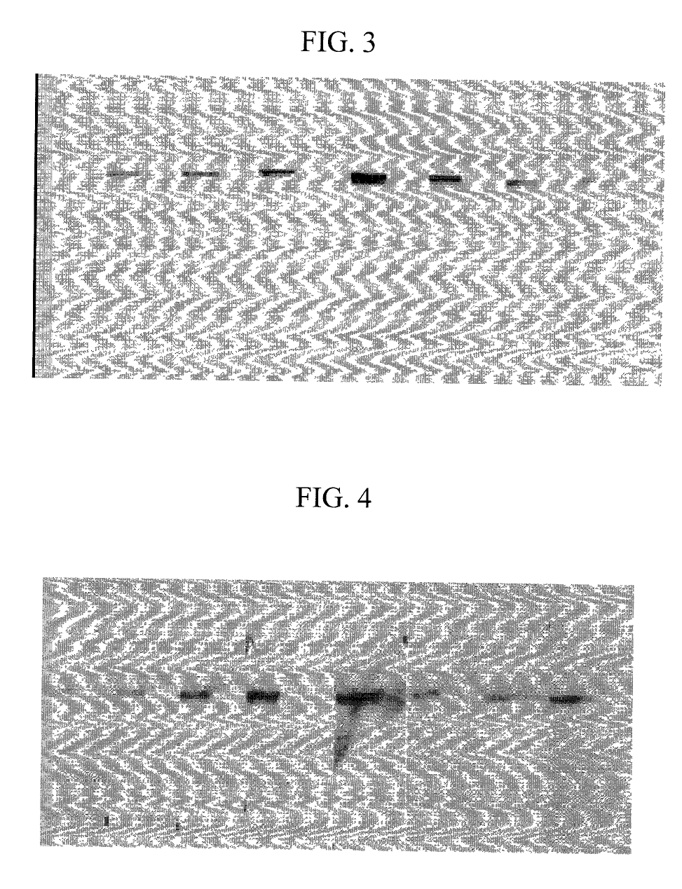

To determine if in vitro transfections can generate sufficient antigen for screening purposes, we transfected murine Hepa and human 293 cells with pCI-LacZ and pCI-Luc vectors. The cells were transfected with 2 μg pDNA per 35-mm well using TransIT-LT1 and TransIT-293, respectively, using the recommended transfection protocol (Mirus). Cells were washed twice with PBS, and resuspended in sample buffer (10⁷ cells/ml). Samples were run into NuPAGE Tris-Acetate gels and blotted onto Hybond-P membranes. The membranes were incubated with a 1:2,000 dilution of serum form a mouse immunized with pCI-Luc (tail vein gene delivery for prime and boost, 3 weeks apart; serum collected 14 days after boost). Following antibody binding, the blots were washed and incubated with a HRP-labeled goat-anti-mouse IgG. Specific binding was detected by chemiluminescent development (FIG. 2). We had anticipated higher background staining in human 293 cells compared with the murine Hepa cells. Yet, both blots only showed signal in a single band in the lanes loaded with luciferase transfected cells. This indicates that transfection can generate sufficient amounts of antigen to allow for screening, that genetic immunization via IV route can result in high-titer antibodies after a single prime-boost cycle, and that Western blotting is an appropriate and relatively simple method to screen for the presence of antigen-specific antibodies in sera of genetically immunized animals.

### EXAMPLE 8

To determine if Western blotting can be used to screen for the presence of antibodies in the serum of genetically immunized animals, 293 cells were transfected with either with pMIR 48 (expressing luciferase) or with control plasmid DNA. Eight mice were immunized with pMIR48 (xx μg, intravascular delivery into the tail vein). After 3 weeks, the mice were boosted with an xx μg pMir48. Boosting was repeated 14 and 28 days later. Cell extracts from the transfected 293 cells were used in a Western blotting experiment as described in example 7. Replicate blots were probed with sera obtained from the immunized mice (diluted 1:100). The results are shown in FIG. 3 (prime+2 boosts) and FIG. 4 (prime+3 boosts).

We claim:

1. A genetic immunization method for inducing an antigen-specific immune response, comprising: a nucleic acid sequence encoding a peptide containing at least one antigenic determinant, operatively linked to one or more control sequences such that the nucleic acid sequence is expressed in a host cell, wherein the nucleic acid sequence is optionally formulated into a particle by complexation with one or more polymers, and wherein the nucleic acid is delivered to a vertebrate host cell.

2. The method of claim 1, wherein the host cell is a lymphoid cell.

3. The method of claim 2, wherein the host cell is a gut-associated lymphoid cell.

4. The method of claim 2, wherein the host cell is a nasal lymphoid cell.

5. The method of claim 1, wherein the delivery step is through intravascular administration.

6. The method of claim 1, wherein the delivery step is through oral administration.

7. The method of claim 1, wherein the nucleic acid is further protected by a coating.

8. The method of claim 7, wherein the coating is an enteric coating.

9. The method of claim 8, wherein the coated nucleic acid is orally delivered.

10. The method of claim 1, wherein the sequence is a DNA sequence.

11. The method of claim 10, wherein the DNA sequence is a plasmid.

12. The method of claim 1, wherein the host is a mammal.

13. A genetic immunization composition formulated for inducing an antigen-specific immune response, comprising: a nucleic acid sequence encoding a peptide containing at least one antigenic determinant, operatively linked to one or more control sequences such that the nucleic acid sequence is expressible in a host cell, wherein the nucleic acid sequence is optionally formulated into a particle by complexation with a polymer, for delivery to a vertebrate host cell.

14. The composition of claim 13, wherein the host cell is a lymphoid cell.

15. The composition of claim 14, wherein the host cell is a gut-associated lymphoid cell.

16. The composition of claim 14, wherein the host cell is a nasal lymphoid cell.

17. The composition of claim 13, wherein the delivery step is through intravascular administration.

18. The composition of claim 13, wherein the delivery step is through oral administration.

19. The composition of claim 13, wherein the nucleic acid is further protected by a coating.

20. The composition of claim 19, wherein the coating is an enteric coating.

21. The composition of claim 20, wherein the coated nucleic acid is orally delivered.

22. The composition of claim 13, wherein the sequence is a DNA sequence.

23. The composition of claim 22, wherein the DNA sequence is a plasmid.

24. The composition of claim 13, wherein the host is a mammal.

25. A method for generating an antibody response in a vertebrate host comprising of administering a nucleic acid encoding an antigen, the nucleic acid optionally being complexed to a polymer, in an amount sufficient to induce the desired immune response directed against the expressed antigen.

26. A method for generating a cellular immune response in a vertebrate host comprising of administering a nucleic acid encoding an antigen, the nucleic acid optionally being complexed to a polymer, in an amount sufficient to induce the desired immune response directed against the expressed antigen.

27. A method for generating an immune response in a vertebrate host, comprising: administering a nucleic acid encoding an antigen, the nucleic acid optionally being complexed to a polymer, in an amount sufficient to induce the desired immune response directed against the expressed antigen, and the nucleic acid is delivered to the intestinal lumen.

28. A method for determining the presence of a genetic immune response in a vertebrate, wherein the antigen is produced in a second vertebrate following nucleic acid delivery.

29. A method for determining the presence of a genetic immune response in a vertebrate, wherein the antigen is produced in a cell line following nucleic acid delivery.

30. A method for determining the presence of a genetic immune response in a vertebrate, wherein the antigen is produced in a primary cell culture following nucleic acid delivery.

31. A kit for genetic immunization, the kit comprising a transfection complex for in vivo gene transfer.

32. A kit for the detection of a genetic immune response, the kit comprising a transfection complex for in vitro gene transfer.

33. A kit for genetic immunization and detection of a genetic immune response, the kit comprising transfection complexes for in vivo and in vitro gene transfer.
